(19) United States
(12) Patent Application Publication
(10) Pub. No.: US 2004/0166540 A1
(43) Pub. Date: Aug. 26, 2004

(54) Methods of detecting CD34 positive and negative hematopoietic stem cells in human samples

(75) Inventors: Fu-Sheng Wang, Lake Zurich, IL (US); Mark Dorfner, Wonder Lake, IL (US); Kojiro Hirai, Nishi-ku (JP); Yukio Hamaguchi, Akashi (JP)

Correspondence Address: Gregory H. Zayia, Brinks Hofer Gilson & Lione, NBC Tower, Suite 3600, P.O. Box 10395, Chicago, IL, 60610, US

(73) Assignee: Sysmex Corporation

(21) Appl. No.: 10/374,278

(22) Filed: Feb. 24, 2003

(51) Int. Cl. G01N 33/567

(52) U.S. Cl. 435/7.21

(57) ABSTRACT

Methods of detecting CD34 negative hematopoietic stem cells in a blood sample are described that include (a) removing CD34 positive cells from the blood sample thereby producing a modified blood sample; (b) extracting CD133 positive cells from the modified blood sample thereby producing a fraction comprising CD34 negative hematopoietic stem cells; and (c) analyzing the fraction with an automated hematology analyzer, thereby detecting the CD34 negative hematopoietic stem cells.

### BACKGROUND

The present invention relates to methods of detecting CD34 negative hematopoietic stem cells in human samples, such as blood and bone marrow. More particularly, the present invention relates to methods of detecting hematopoietic stem cells, including CD34 negative hematopoietic stem cells, with an automated hematology analyzer.

In clinical medicine, and particularly in hematopoietic stem cell (HSC) transplantations, the cell surface marker CD34 has been employed for the selection and detection of stem cells. Recent studies indicate that hematopoietic stem cells (HSCs) may be CD34 positive (CD34⁺) or CD34 negative (CD34⁻). As shown in FIG. 1, CD34 expression is reversible and varies according to clinical circumstances. Currently, it is thought that CD34⁻ HSCs are capable of self-renewal and interconversion to CD34⁺ HSCs. Moreover, recent studies indicate that the earliest HSC population likely corresponds to CD34⁻ cells, which can differentiate into CD34⁺ cells circulating in the blood triggering their return to the bone marrow and repopulating the progenitor cells.

Inasmuch as CD34⁻ HSCs appear to lie at an earlier stage in cell development than their positive counterparts, they are of increasing interest for their potentially high degree of plasticity (i.e., the ability of an adult stem cell from one type of tissue to give rise to specialized cells and/or other types of tissue or organs). However, due to the absence of positive markers on CD34⁻ cells, there is difficulty involved in attempting to employ them for stem cell selection and detection.

Conventional methods for measuring CD34⁺ HSCs—in addition to being expensive, laborious, and time consuming—do not provide measurements of CD34⁻ HSCs. For example, conventional flow cytometry involves the use of fluorescently labeled CD34⁺ antibodies in complicated, multi-step procedures requiring highly skilled technicians and painstaking data analysis; unfortunately, measurements of CD34⁻ HSCs are not included in the final counts.

The measurement of both CD34⁺ and CD34⁻ HSCs is important in order to obtain more accurate information about the total hematopoietic cell pool. Such knowledge facilitates clinical diagnoses, determination of correct clinical treatments, and selection of optimum times for stem cell collection.

Knowledge of the total number of HSCs is particularly important in connection with stem cell transplantations. In spite of this importance, HSCs have traditionally been measured based solely on CD34⁺ markers, resulting in incomplete and inaccurate information profiles of the hematopoiesis in a given patient.

### SUMMARY

The scope of the present invention is defined solely by the appended claims, and is not affected to any degree by the statements within this summary.

Briefly stated, a first method of detecting CD34 negative hematopoietic stem cells in a blood sample embodying features of the present invention includes (a) removing CD34 positive cells from the blood sample thereby producing a modified blood sample; (b) extracting CD133 positive cells from the modified blood sample thereby producing a fraction comprising CD34 negative hematopoietic stem cells; and (c) analyzing the fraction with an automated hematology analyzer, thereby detecting the CD34 negative hematopoietic stem cells.

A second method of detecting CD34 negative hematopoietic stem cells in a blood sample embodying features of the present invention includes (a) treating the blood sample with CD34 antibodies to produce a modified blood sample substantially depleted of CD34 positive cells; (b) treating the modified blood sample with CD133 antibodies to obtain a fraction comprising CD133 positive cells and CD34 negative hematopoietic stem cells; and (c) analyzing the fraction with an automated hematology analyzer, thereby detecting the CD34 negative hematopoietic stem cells.

A method of detecting CD34 hematopoietic stem cells in a blood sample embodying features of the present invention includes: (a): treating the blood sample with CD34 antibodies to produce a modified blood sample substantially depleted of CD34 positive cells and a fraction of the blood sample comprising CD34 positive cells removed from the blood sample; (b) treating the modified blood sample with CD133 antibodies to obtain a fraction of the modified blood sample comprising CD133 positive cells and CD34 negative hematopoietic stem cells; (c) analyzing the fraction of the blood sample with an automated hematology analyzer, thereby detecting the CD34 positive cells; and (d) analyzing the fraction of the modified blood sample with an automated hematology analyzer, thereby detecting the CD34 negative hematopoietic stem cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram representing current understanding of the relationship between CD34⁻ HSCs, CD34⁺ cells, committed blood cells, and mature blood cells partitioned between the bone marrow and peripheral blood of a patient.

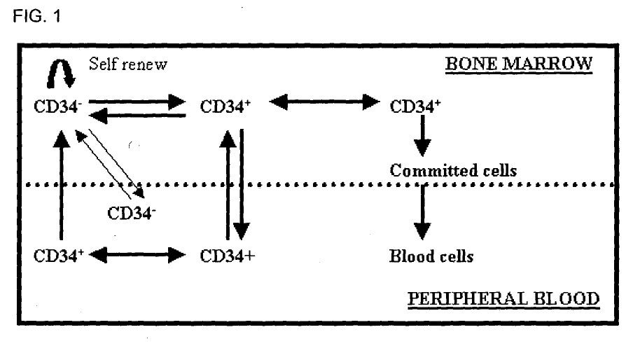

FIG. 2 shows a flow chart illustrating representative acts in a method embodying features of the present invention.

FIG. 3 is a flow cytometry graph of G-CSF mobilized blood stained with FITC-labeled and PE-labeled control antibodies.

FIG. 4 is a flow cytometry graph of G-CSF mobilized peripheral blood stained with FITC-labeled CD34 and PE-labeled CD133.

FIG. 5 is a flow cytometry graph of a cell sample depleted of CD34⁺ cells with CD34 magnetic beads, and then purified with CD133 magnetic beads (i.e., a cell sample containing CD34 negative HSCs).

FIG. 6 shows a two-dimensional scattergram of an IMI channel with the HPC detection area highlighted.

FIG. 7 shows a two-dimensional scattergram of a G-CSF mobilized peripheral blood sample tested on a SYSMEX SE-9000 automated hematology analyzer.

FIG. 8 shows a two-dimensional scattergram of cells removed from the G-CSF mobilized peripheral blood sample of FIG. 7 with CD34 antibodies.

FIG. 9 shows a two-dimensional scattergram of cells extracted from a CD34 depleted modified blood sample with CD133 antibodies.

### DETAILED DESCRIPTION OF THE PRESENTLY PREFERRED EMBODIMENTS

Rapid, facile, and inexpensive methods enabling detection of both CD34⁺ and CD34⁻ HSCs, which may be advantageously employed in connection with clinical diagnoses and treatments as well as future applications in stem cell regenerative medicine, have been discovered and are described hereinbelow.

By way of introduction, a representative method of detecting CD34⁻ HSCs with an automated hematology analyzer in accordance with the present invention may be described as follows: CD34 positive cells are depleted from a G-CSF mobilized human peripheral blood sample by an immunologic method (e.g., incubation with labeled C34 antibody magnetic beads followed by magnetic separation). The CD34⁻ cells thus obtained are purified (e.g., by incubation with labeled CD133 antibody magnetic beads followed by magnetic separation from the CD34-depleted sample). The purified CD34⁻ and CD133⁺ cells are the above-mentioned CD34⁻ HSCs. The cells are then tested by flow cytometry to evaluate cell type and purity, and by automated hematology analysis. Automated hematology analysis shows that the CD34⁻ HSCs are located in the same HPC region of the scattergram as the corresponding CD34⁺ cells, thus confirming that an automated hematology analyzer can be used to measure not only CD34⁺ cells but also CD34⁻ HSCs. Accordingly, methods embodying features of the present invention are likely to provide increased understanding of stem cell measurements in general in addition to providing total stem cell counts that include both the number of CD34⁺ cells and the number of CD34⁻ HSCs present in a sample.

Throughout this description and in the appended claims, the following definitions are to be understood:

The term “CD133⁺ cell” refers to hematopoietic stem/progenitor cells, which may be CD34 positive or CD34 negative.

The term “CD34⁻ cell” refers to cells without a CD34 marker, which may include CD34 negative HSCs and more mature (non-stem progenitor cells) cells.

The term “CD34⁻ HSC” refers to hematopoietic stem cells without a CD34 marker, which may be CD133 positive or negative.

The term “antibody” refers to a specific protein capable of binding an antigen or portion thereof. The generic term “antibody” subsumes polyclonal antibodies, monoclonal antibodies and antibody fragments.

The term “antigen” refers to any substance capable of binding to an antibody.

The term “CD133 antibody” refers to an antibody also known as AC133, which is a marker for human hematopoietic stem and progenitor cells. The terminology “CD133” was introduced at the 7^{th} International Workshop and Conference on Human Leukocyte Differentiation Antigens (2000).

The term “label” refers to an identifying tag that can be attached to a carrier substance or molecule to facilitate detection. A label may be attached to its carrier substance directly or indirectly by means of a linking or bridging moiety. Suitable labels include but are not limited to enzymes (e.g., β-galactosidase, peroxidase, etc.), fluorescent compounds (e.g., rhodamine, fluorescein isothiocyanate or FITC, etc.), luminescent compounds (e.g., dioxetanes, luciferin, etc.), radioactive isotopes (e.g., ¹²⁵I), protein-binding partners (e.g., biotin), and the like, and combinations thereof.

The terms “detecting,” “detection,” “analyzing,” and “analysis” (e.g., “detecting CD34 negative hematopoietic stem cells,” etc.) refer to any quantitative, semi-quantitative, or qualitative method for determining an analyte in general, and a CD34 cell in particular. For example, a method that merely detects the presence or absence of a CD34 cell in a sample lies within the scope of the present invention, as do methods that provide data as to the amount or concentration of the cells in the sample.

The terms “depleting” and “removing” refer to the removal of a majority (i.e., more than one-half) of a particular type of cell (e.g., CD34⁺) from a sample.

A first method embodying features of the present invention includes (a) removing CD34⁺ cells (e.g., CD34⁺ single positive cells, CD34⁺/CD133⁺ double positive cells) from a blood sample thereby producing a modified blood sample; (b) extracting CD133⁺ cells from the modified blood sample thereby producing a fraction comprising CD34⁻ hematopoietic stem cells; and (c) analyzing the fraction with an automated hematology analyzer, thereby detecting the CD34⁻ hematopoietic stem cells.

The removal of CD34⁺ cells from a blood sample in accordance with the present invention is preferably accomplished using an immunologic technique and, more preferably, involves treating the blood sample with CD34 antibodies. Preferably, the CD34 antibodies are attached to magnetic beads, which enable facile separation of CD34⁺ cells from blood samples using a magnetic cell separator. Preferred magnetic beads for use in accordance with the present invention include the super-paramagnetic micro-beads sold by Miltenyi Biotec Inc of Auburn, Calif., and the substantially uniform, super-paramagnetic, mono-disperse polymer beads sold under the tradename DYNANEADS by Dynal Biotech ASA of Oslo, Norway. Preferred magnetic cell separators the automated magnetic cell selection system sold under the tradename ISOLEX by Nexell Therapeutics, Inc. of Irvine, Calif.

The CD34⁺ cells thus removed from the blood sample may be detected or analyzed directly, or first separated from the magnetic beads to which they are attached by treatment with an appropriate enzyme (e.g., chymopapain). The CD34⁺ cells thus recovered from the magnetic beads may be then be detected and/or analyzed (e.g., via flow cytometry, automated hematology analysis, or the like).

In presently preferred embodiments in accordance with the present invention, the CD34 antibodies are labeled (e.g., with a fluorescent label), which facilitates detection and analysis of the removal of CD34⁺ cells from a blood sample (e.g., via flow cytometry, automated hematology analysis, or the like). An especially preferred label for CD34 antibodies is fluorescein isothiocyanate (FITC).

The extraction of CD133⁺ cells from a modified blood sample (i.e., a blood sample from which CD34⁺ cells have been previously depleted) in accordance with the present invention is preferably accomplished using an immunologic technique and, more preferably, involves treating the blood sample with CD133 antibodies. Preferably, the CD133 antibodies are attached to magnetic beads from Miltenyi Biotec Inc., as described above, which enables facile extraction of CD133⁺ cells from the modified blood samples using a magnetic cell separator (e.g., such as an ISOLEX system, as described above).

The cells thus extracted from the modified blood sample are referred to herein as “purified cells” or “cells purified with CD133,” and include CD133⁺ and CD34⁻ cells. Inasmuch as the cell samples that are subjected to CD133 purification have already been substantially depleted of CD34⁺ cells, the cells that are ultimately extracted in the purification process include CD34⁻ cells. Moreover, inasmuch as the CD133 antibodies recognize HSCs, the cells that are ultimately extracted from the CD34⁺-depleted modified blood sample include CD34⁻ HSCs.

The purified cells thus extracted from the modified blood sample may be detected or analyzed directly. In addition, it is presently preferred that CD133 antibodies used in accordance with the present invention be labeled (e.g., with a fluorescent label) to facilitate analysis and detection of the purified cells extracted from the blood sample (e.g., via flow cytometry, automated hematology analysis, or the like). An especially preferred label for CD133 antibodies is R-phycoerythrin (PE).

In a first series of presently preferred embodiments, the analysis of the purified fraction comprising CD34⁻ HSCs and the analysis of the CD34⁺ cells removed from the original blood sample are preferably conducted on automated hematology analyzers. Automatic blood cell analyzers equipped with channels capable of detecting immature leukocytes (i.e., immature leukocyte information or IMI channels) are especially preferred at present. Automated analyzers suitable for use in accordance with the present invention include the multiparameter analyzers XE2100 and SE-9000 sold by the Sysmex Corporation of Kobe, Japan. In a related series of presently preferred embodiments, the results of these analyses are confirmed through parallel flow cytometry analyses.

A blood sample for use in accordance with the present invention preferably comprises cells selected from the group consisting of peripheral blood cells, bone marrow blood cells, cord blood cells, and combinations thereof. Cells from peripheral blood that has been mobilized by granulocyte colony stimulating factor (G-CSF) are especially preferred at present. For G-CSF mobilization in a normal donor, the procedure is to first administer G-CSF to the donor, and afterwards to collect mononucleated cells in the peripheral blood and isolate stem cells from the mononucleated cells if necessary.

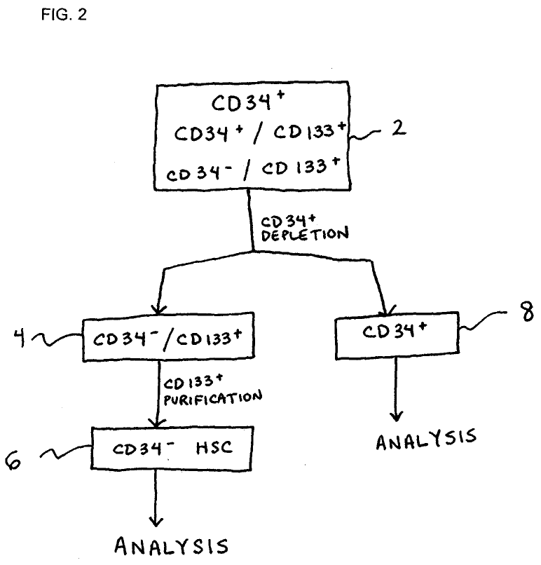

Turning now to the drawings, FIG. 2 shows a schematic outline of representative acts embodying features of the present invention. Initially, CD34⁺ cells are depleted from a blood sample 2 (e.g., G-CSF mobilized peripheral blood cells) to produce a modified blood sample 4 (e.g., by an immunologic method such as treatment with CD34 antibodies). CD133⁺ cells are then extracted from the CD34⁺-depleted modified blood sample 4 (i.e., modified blood sample 4 is “purified” with CD133 antibodies) to produce a fraction 6 comprising CD34⁻ hematopoietic stem cells. Finally, fraction 6 is analyzed and the CD34⁻ hematopoietic stem cells contained therein are detected. In alternative embodiments, the CD34⁺ cells 8 removed from blood sample 2 are also analyzed, such that a complete measurement of the CD34 cells in the original sample 2, which includes both CD34⁺ and CD34⁻ HSCs, is obtained. In such cases, it is presently preferred that the two portions 6 and 8 be tested on a flow cytometer as well as an automated hematology analyzer.

The manner and process of detecting CD34⁺ in cell samples by means of automated hematology analyzers is known to those of ordinary skill in the art and described in literature references such as U.S. Pat. No. 5,830,701 to Houwen et al. and “Principles of Measurement in Hematology Analyzers Manufactured by Sysmex Corporation” by Keiji Fujimoto (Sysmex Journal International, 1999, 9, No. 1, pp. 31-44). Both of these references are incorporated herein by reference in their entireties, except that in the event of any inconsistent disclosure or definition from the present application, the disclosure or definition herein shall be deemed to prevail.

Suitable methods of cell detection for use in accordance with the present invention include methods based on electric capacitance, electric impedance, light scattering, and the like, and combinations thereof. The RF/DC method of detection whereby an electric impedance (DC) method and an electric capacitance (RF) method are applied in order to collect information regarding cells size and cell contents, respectively, is especially preferred at present.

The following representative methods embodying features of the present invention for detecting CD34⁻ HSCs are provided solely by way of illustration, and are not intended to limit the scope of the appended claims or their equivalents.

### EXAMPLES

In the examples that follow, cells from human G-CSF mobilized peripheral blood are employed, but it is to be understood that cells obtained from alternative sources including but not limited to bone marrow and cord blood may be employed instead. Purified cells are observed by automated hematology analyzers such as the SYSMEX SE-9000 and XE-2100 and, in control experiments, by flow cytometry using fluorescence labeled antibodies to determine cell types and purity at different stages in purification.

Mononucleated G-CSF mobilized peripheral blood cells are obtained from volunteer donors. The blood cells are mixed with labeled CD34 antibody magnetic beads and incubated at 4 degrees Celsius for 20 minutes with gentle mixing. CD34⁺ cells are removed from the CD34⁻ cells present in the sample via a magnetic separation procedure. The CD34-depleted modified blood sample is then mixed with labeled CD133 antibody magnetic beads and incubated for 20 minutes. CD133⁺ cells (i.e., CD34⁻ cells) are purified from the CD34-depleted modified blood sample via a magnetic separation procedure. The CD34⁺ cells and the CD133⁺ cells purified by the procedures outlined above are tested on a flow cytometer and on an automated hematology analyzer.

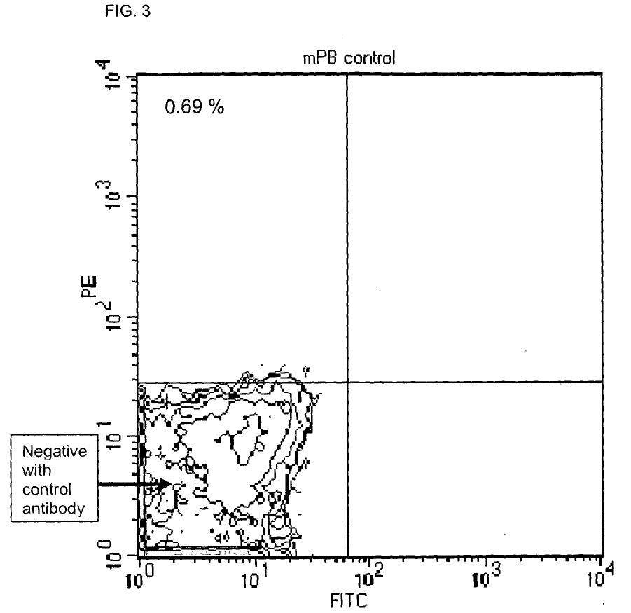

The above-described representative procedures will be further illustrated with reference to FIGS. 3-10

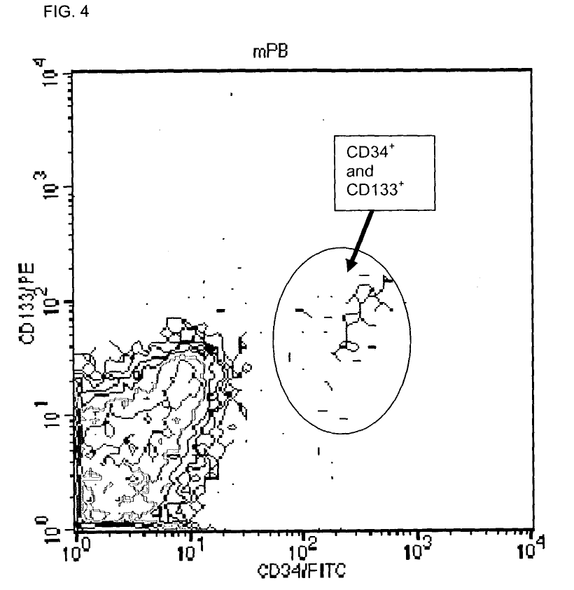

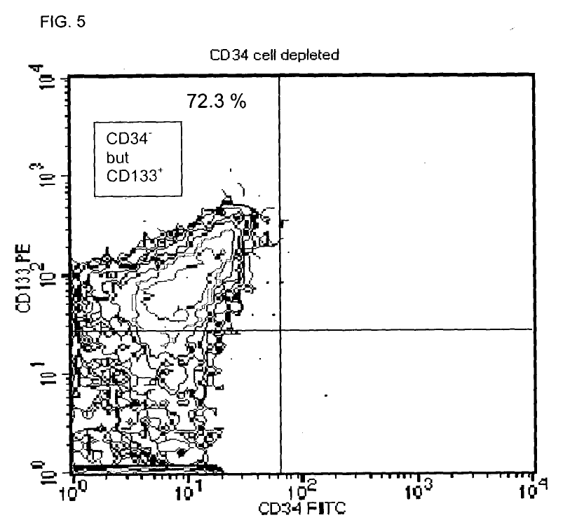

FIG. 3 shows a flow cytometry graph of G-CSF mobilized blood cells prior to purification, which are stained with FITC-labeled and PE-labeled control antibodies. As shown in FIG. 3, no positive cells are observed when either FITC-labeled or PE-labeled control antibodies are used for the staining (as based on the relative intensities of fluorescence compared with control). The cells showed high intensity relative to the positive cells of FIGS. 4 and 5 (described below) under the same flow cytometer settings. The CD133⁺ cells in the upper right correspond to 0.69%.

FIG. 4 shows a flow cytometry graph of G-CSF mobilized peripheral blood cells prior to purification, which are stained with FITC-labeled CD34 and PE-labeled CD133 antibodies. The circled region represents a “double positive” cell population that is labeled with both antibodies. A small population of individual CD34⁺ cells is observed in the lower right corner of the graph and a small population of CD133⁺ cells is observed in the upper left corner of the graph.

FIG. 5 shows a flow cytometry graph of a cell sample depleted of CD34⁺ cells with CD34 antibody magnetic beads, and then purified with CD133 antibody magnetic beads. The purified cells thus obtained correspond to CD34⁻ HSCs, which are detected with an automated hematology analyzer. The cells in the upper left of FIG. 5 are CD133⁺/CD34⁻ cells, the cells in the lower right are CD34⁺/CD133⁻ cells, and the cells in the upper right are double positive CD34⁺/CD133⁺ cells. As shown in FIG. 5, the CD133 positive cells are significantly enriched (72.3%) as compared to FIGS. 3 and 4, whereas CD34⁺ positive cells have essentially disappeared.

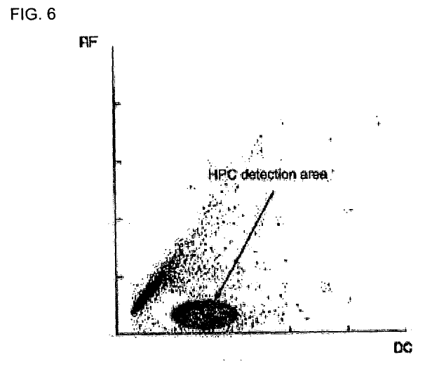

FIG. 6 shows a two-dimensional scattergram corresponding to the IMI channel. The axes of the scattergram correspond to DC signal intensity and RF signal intensity. The HPC detection area is indicated in the oval area.

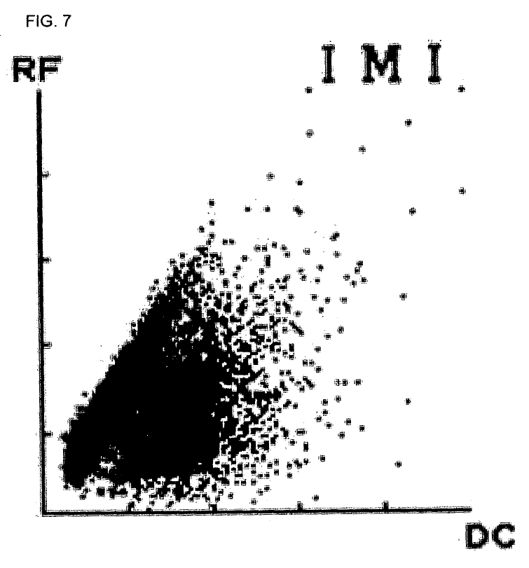

FIG. 7 shows a two-dimensional scattergram of a G-CSF mobilized peripheral blood sample prior to purification tested on a SYSMEX SE-9000 automated hematology analyzer. As shown in FIG. 7, numerous cells are located in the HPC area (light pink) of the IMI channel.

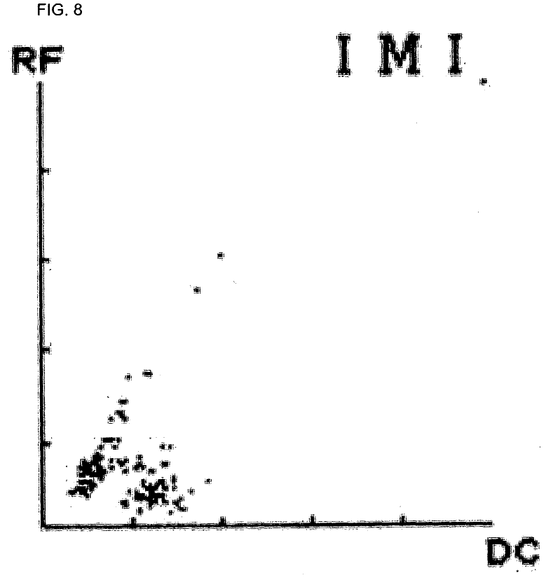

FIG. 8 shows a two-dimensional scattergram of cells removed from the G-CSF mobilized peripheral blood sample of FIG. 7 with CD34 antibodies. As shown in FIG. 7, the majority of cells are located in the narrow HPC region of the graph.

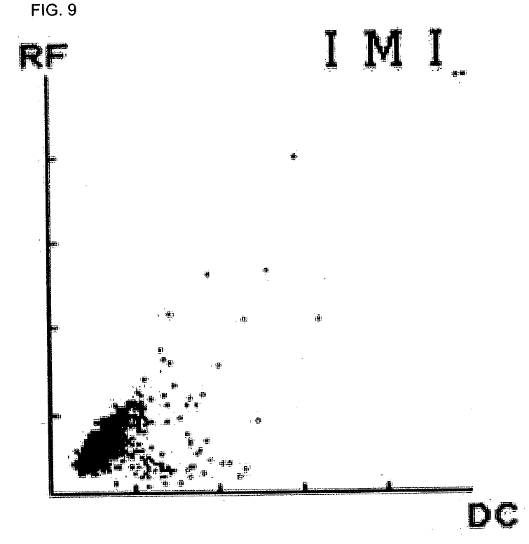

FIG. 9 shows a two-dimensional scattergram of cells extracted from a CD34 depleted modified blood sample with CD133 antibodies. As with the CD34⁺ cells of FIG. 8, the majority of extracted cells shown in FIG. 9 are located in the HPC area of the graph, thus confirming that an automated hematology analyzer can be used to measure CD34⁺ as well as CD34⁻ HSCs.

The total HSC count thus obtained may be useful for the clinical monitoring of hematopoiesis, stem cell mobilization, chemotherapy, radiation therapy, apheresis, and the like, and combinations thereof.

The foregoing detailed description and examples have been provided by way of explanation and illustration, and are not intended to limit the scope of the appended claims. Many variations in the presently preferred embodiments illustrated herein will be obvious to one of ordinary skill in the art, and remain within the scope of the appended claims and their equivalents.

1. A method of detecting CD34 negative hematopoietic stem cells in a blood sample, comprising:
removing CD34 positive cells from the blood sample thereby producing a modified blood sample;
extracting CD133 positive cells from the modified blood sample thereby producing a fraction comprising CD34 negative hematopoietic stem cells; and
analyzing the fraction with an automated hematology analyzer, thereby detecting the CD34 negative hematopoietic stem cells.

2. The method of claim 1, wherein the removing of the CD34 positive cells comprises treating the blood sample with CD34 antibodies.

3. The method of claim 2, wherein the CD34 antibodies are fluorescently labeled.

4. The method of claim 3, wherein the CD34 antibodies are fluorescently labeled with fluorescein isothiocyanate.

5. The method of claim 1, wherein the removing of the CD34 positive cells comprises treating the blood sample with magnetic beads, each of which is bound to a fluorescently labeled CD34 antibody.

6. The method of claim 5, further comprising separating a portion comprising the CD34 positive cells and the magnetic beads from the modified blood sample.

7. The method of claim 1, wherein the extracting of the CD133 positive cells from the modified blood sample comprises treating the modified blood sample with CD133 antibodies.

8. The method of claim 7, wherein the CD133 antibodies are fluorescently labeled.

9. The method of claim 8, wherein the CD133 antibodies are fluorescently labeled with R-phycoerythrin.

10. The method of claim 1, wherein the extracting of the CD133 positive cells from the modified blood sample comprises treating the modified blood sample with magnetic beads, each of which is bound to a fluorescently labeled CD133 antibody, wherein the fraction further comprises the magnetic beads.

11. The method of claim 10, further comprising separating the fraction comprising the CD34 negative hematopoietic stem cells and the magnetic beads from a residual portion of the modified blood sample.

12. The method of claim 1, wherein the fraction further comprises CD133 positive cells and CD34 positive hematopoietic stem cells.

13. The method of claim 1, wherein the automated hematology analyzer comprises a SYSMEX SE-9000 analyzer.

14. The method of claim 1, wherein the automated hematology analyzer comprises a SYSMEX XE-2100 analyzer.

15. The method of claim 1, further comprising detecting the CD34 positive cells removed from the blood sample.

16. The method of claim 15, wherein the CD34 positive cells are detected with an automated hematology analyzer.

17. The method of claim 16, wherein the automated hematology analyzer comprises a SYSMEX SE-9000 analyzer.

18. The method of claim 16, wherein the automated hematology analyzer comprises a SYSMEX XE-2100 analyzer.

19. The method of claim 1, wherein the blood sample comprises cells selected from the group consisting of peripheral blood cells, bone marrow cells, cord blood cells, and combinations thereof.

20. The method of claim 1, wherein the blood sample comprises G-CSF mobilized peripheral blood cells.

21. The method of claim 1, further comprising producing a scattergram comprising a hematopoietic progenitor cell region, wherein a presence of the CD34 negative hematopoietic stem cells is identifiable therein.

22. The method of claim 1, wherein the detecting of the CD34 negative hematopoietic stem cells in the blood sample contributes to clinical monitoring of a treatment selected from the group consisting of hematopoiesis, stem cell mobilization, chemotherapy, radiation therapy, apheresis, and combinations thereof.

23. A method of detecting CD34 negative hematopoietic stem cells in a blood sample, comprising:
treating the blood sample with CD34 antibodies to produce a modified blood sample substantially depleted of CD34 positive cells;
treating the modified blood sample with CD133 antibodies to obtain a fraction comprising CD133 positive cells and CD34 negative hematopoietic stem cells; and
analyzing the fraction with an automated hematology analyzer, thereby detecting the CD34 negative hematopoietic stem cells.

24. A method of detecting CD34 hematopoietic stem cells in a blood sample, comprising:
treating the blood sample with CD34 antibodies to produce a modified blood sample substantially depleted of CD34 positive cells and a fraction of the blood sample comprising CD34 positive cells removed from the blood sample;
treating the modified blood sample with CD133 antibodies to obtain a fraction of the modified blood sample comprising CD133 positive cells and CD34 negative hematopoietic stem cells;
analyzing the fraction of the blood sample with an automated hematology analyzer, thereby detecting the CD34 positive cells; and
analyzing the fraction of the modified blood sample with an automated hematology analyzer, thereby detecting the CD34 negative hematopoietic stem cells.
